(19) United States
(12) Patent Application Publication
(10) Pub. No.: US 2024/0153596 A1
(43) Pub. Date: May 9, 2024

(54) ARTIFICIAL INTELLIGENCE-BASED MOLECULE PROCESSING METHOD AND APPARATUS, ELECTRONIC DEVICE, COMPUTER-READABLE STORAGE MEDIUM, AND COMPUTER PROGRAM PRODUCT

(71) Applicant: TENCENT TECHNOLOGY (SHENZHEN) COMPANY LIMITED, Shenzhen (CN)

(72) Inventors: Geyan YE, Shenzhen (CN); Wei LIU, Shenzhen (CN)

(73) Assignee: TENCENT TECHNOLOGY (SHENZHEN) COMPANY LIMITED, Shenzhen (CN)

(21) Appl. No.: 18/417,891

(22) Filed: Jan. 19, 2024

Related U.S. Application Data
(63) Continuation of application No. PCT/CN23/96778, filed on May 29, 2023.

(30) Foreign Application Priority Data
Aug. 16, 2022 (CN) 202210980553.2

(51) Int. Cl. G16C 20/20

(52) U.S. Cl. CPC G16C 20/20

(57) ABSTRACT

An artificial intelligence-based molecule processing method including obtaining a three-dimensional structure of a target molecule, calling a neural network model to perform energy error prediction processing on the three-dimensional structure of the target molecule to obtain an energy error of the target molecule; the neural network model being trained by fitting an energy error of a sample molecule, performing the first energy computation processing on the three-dimensional structure of the target molecule to obtain first energy of the target molecule; and performing error correction processing on the first energy of the target molecule based on the energy error of the target molecule to obtain second energy of the target molecule.

### CROSS-REFERENCE TO RELATED APPLICATIONS

This application is a continuation application of International Application No. PCT/CN2023/096778 filed on May 29, 2023, which claims priority to Chinese Patent Application No. 202210980553.2 filed with the China National Intellectual Property Administration on Aug. 16, 2022, the disclosures of each being incorporated by reference herein in their entireties.

### FIELD

The disclosure relates to artificial intelligence technologies, and in particular, to an artificial intelligence-based molecule processing method and apparatus, an electronic device, a computer-readable storage medium, and a computer program product.

### BACKGROUND

Artificial intelligence (AI) is a comprehensive technology of computer science. By studying design principles and implementation methods of various intelligent machines, AI makes the machines have functions of perception, inference, and decision-making. The artificial intelligence technology is an integrated discipline, covering a wide range of fields, such as natural language processing technology and machine learning/deep learning. With the development of technologies, the artificial intelligence technology will be applied in more fields and play more and more important values.

In a new drug research and development process and a material development process after target identification and validation are completed, a candidate drug compound needs to be screened. In a screening process, energy of a molecule generally needs to be computed in a scenario such as computation of molecular properties. A computation result is helpful for drug research and development personnel to analyze molecular properties, binding capabilities of molecules and protein pockets, and the like. In the related art, molecular energy is generally obtained in a computational chemistry manner, in which precision of obtaining molecular energy and a speed of obtaining molecular energy cannot be both considered.

### SUMMARY

Some embodiments provide an artificial intelligence-based molecule processing method and apparatus, an electronic device, a computer-readable storage medium, and a computer program product, which can simultaneously improve precision and increase a speed of molecular energy computation.

Some embodiments provide an artificial intelligence-based molecule processing method, performed by an electronic device and including: obtaining a three-dimensional structure of a target molecule; calling a neural network model to perform energy error prediction processing on the three-dimensional structure of the target molecule to obtain an energy error of the target molecule, the neural network model being trained by fitting an energy error of a sample molecule; performing the first energy computation processing on the three-dimensional structure of the target molecule to obtain first energy of the target molecule; and performing error correction processing on the first energy of the target molecule based on the energy error of the target molecule to obtain second energy of the target molecule, wherein the energy error of the sample molecule is a difference between computation results obtained based on energy of the sample molecule is separately computed according to two energy computation mechanisms, the two energy computation mechanisms comprising first energy computation processing and second energy computation processing, and wherein precision of the first energy computation processing is less than precision of the second energy computation processing and a speed of the first energy computation processing is greater than a speed of the second energy computation processing.

Some embodiments provide an artificial intelligence-based molecule processing apparatus, including: at least one memory configured to store program code; and at least one processor configured to read the program the program code comprising: obtaining code configured to cause at least one of the at least one processor to obtain a three-dimensional structure of a target molecule; neural network code configured to cause at least one of the at least one processor to call a neural network model to perform energy error prediction processing on the three-dimensional structure of the target molecule, to obtain an energy error of the target molecule, the neural network model being trained by fitting an energy error of a sample molecule; computation code configured to cause at least one of the at least one processor to perform the first energy computation processing on the three-dimensional structure of the target molecule to obtain first energy of the target molecule; and correction code configured to cause at least one of the at least one processor to perform error correction processing on the first energy of the target molecule based on the energy error of the target molecule, to obtain second energy of the target molecule, wherein the energy error of the sample molecule is a difference between computation results obtained based on energy of the sample molecule is separately computed according to two energy computation mechanisms, the two energy computation mechanisms comprising first energy computation processing and second energy computation processing, and wherein precision of the first energy computation processing is less than precision of the second energy computation processing and a speed of the first energy computation processing is greater than a speed of the second energy computation processing.

Some embodiments provide a non-transitory computer-readable storage medium storing computer code which, when executed by at least one processor, causes the at least one processor to at least: obtain a three-dimensional structure of a target molecule; call a neural network model to perform energy error prediction processing on the three-dimensional structure of the target molecule to obtain an energy error of the target molecule, the neural network model being trained by fitting an energy error of a sample molecule; perform the first energy computation processing on the three-dimensional structure of the target molecule to obtain first energy of the target molecule; and perform error correction processing on the first energy of the target molecule based on the energy error of the target molecule to obtain second energy of the target molecule, wherein the energy error of the sample molecule is a difference between computation results obtained based on energy of the sample molecule is separately computed according to two energy computation mechanisms, the two energy computation mechanisms comprising first energy computation processing and second energy computation processing, and wherein precision of the first energy computation processing is less than precision of the second energy computation processing and a speed of the first energy computation processing is greater than a speed of the second energy computation processing.

First energy computation processing is performed on a three-dimensional structure of a target molecule to obtain first energy of the target molecule. Because a speed of first energy computation processing is higher than that of second energy computation processing, a speed of energy computation processing is increased, energy error prediction processing is performed on the three-dimensional structure of the target molecule by using a neural network model to obtain an energy error of the target molecule, and error correction processing is performed on the computed first energy based on the energy error, to obtain second energy of the target molecule. Because the energy error can represent a computation result difference between second energy computation processing with high precision and first energy computation processing with low precision, the computed first energy can be corrected by using an energy error obtained through deep learning prediction, thereby increasing precision of the second energy.

### BRIEF DESCRIPTION OF THE DRAWINGS

To describe the technical solutions of some embodiments of this disclosure more clearly, the following briefly introduces the accompanying drawings for describing some embodiments. The accompanying drawings in the following description show only some embodiments of the disclosure, and a person of ordinary skill in the art may still derive other drawings from these accompanying drawings without creative efforts. In addition, one of ordinary skill would understand that aspects of some embodiments may be combined together or implemented alone.

FIG. 1 is a schematic flowchart of drug research and development according to some embodiments.

FIG. 2 is a schematic architecture diagram of an artificial intelligence-based molecule processing system according to some embodiments.

FIG. 3 is a schematic structural diagram of an electronic device according to some embodiments.

FIG. 4A is a schematic flowchart of an artificial intelligence-based molecule processing method according to some embodiments.

FIG. 4B is a schematic flowchart of an artificial intelligence-based molecule processing method according to some embodiments.

FIG. 4C is a schematic flowchart of an artificial intelligence-based molecule processing method according to some embodiments.

FIG. 5 is a schematic structural diagram of a deep quantum chemical model according to some embodiments.

FIG. 6 is an effect comparison diagram of a first data set according to some embodiments.

FIG. 7 is an effect comparison diagram of a second data set according to some embodiments.

### DESCRIPTION OF EMBODIMENTS

To make the objectives, technical solutions, and advantages of the present disclosure clearer, the following further describes the present disclosure in detail with reference to the accompanying drawings. The described embodiments are not to be construed as a limitation to the present disclosure. All other embodiments obtained by a person of ordinary skill in the art without creative efforts shall fall within the protection scope of the present disclosure and the appended claims.

In the following description, the term “some embodiments” describes subsets of all possible embodiments, but it may be understood that “some embodiments” may be the same subset or different subsets of all the possible embodiments, and can be combined with each other without conflict. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. For example, the phrase “at least one of A, B, and C” includes within its scope “only A”, “only B”, “only C”, “A and B”, “B and C”, “A and C” and “all of A, B, and C.”

In the following description, the term “first\second\third” is merely used for distinguishing between similar objects, and does not represent a specific sorting for the objects. It may be understood that a specific sequence or an order of “first\second\third” may be interchanged when allowed, so that the embodiments described herein can be implemented in a sequence other than that shown or described herein.

Unless otherwise defined, meanings of all technical and scientific terms used herein are the same as those usually understood by a person skilled in the art. The terms used herein are merely intended to describe some embodiments, and are not intended to be limiting.

Before embodiments are further described in detail, a description is made on terms in some embodiments, and the terms in some embodiments are applicable to the following explanations.

1) Computational chemistry: A branch of theoretical chemistry that uses effective mathematical approximation and computer programs to compute molecular properties, such as total energy, dipole moment, quadrupole moment, vibration frequency, and reaction activity, to explain some specific chemical problems.

2) Molecular energy: Molecular energy includes kinetic energy and potential energy of a molecule. Molecular kinetic energy refers to energy of a molecule due to motion, and molecular potential energy refers to potential energy generated by a molecular force of a molecule and determined by a relative position.

3) Semi-empirical quantum mechanical methods: A quantum mechanical method is highly accurate in theory, but in the case of a biological macromolecule system, computation costs are very high, and cannot meet an actual application requirement. Semi-empirical quantum mechanical methods in which an empirical parameter is introduced is approximate methods, which make a tradeoff between time and accuracy, can be used for scoring and estimating affinity between a ligand and a protein, and are of great significance in computer-aided drug design.

4) Delta learning: Delta learning means that a learning system can continuously learn new knowledge from new samples and preserve most previously learned knowledge. Delta learning is very similar to human learning patterns.

5) The ab initio method refers to a quantum chemical computation method for directly solving the Schrödinger equation based on a basic principle of quantum mechanics in quantum chemistry computation. The ab initio method is characterized by no empirical parameters and no excessive simplification of a system. Various different chemical systems are computed in basically the same way.

6) Density functional theory: is a method for studying electronic structures of a multi-electronic system. The density functional theory is widely used in physics and chemistry, especially in studying properties of molecules and condensed states. It is one of the most commonly used methods in the field of condensed matter physics computational materials science and computational chemistry.

Molecular energy computation manners in the related art mainly include two manners: based on computational chemistry and based on deep learning. The computational chemistry manner includes a manner of quantum mechanics and a manner of molecular mechanics (MM).

The manner of quantum mechanics includes the ab initio method and semi-empirical quantum mechanical methods. The ab initio method is based on the first principle of quantum chemistry, and uses a rigorous approximation to solve the Schrödinger equation. In the ab initio method, there is a manner based on a wave function and a manner based on density functional. A representative manner based on a wave function is a manner based on the Hartree-Fock Equation, and a representative manner based on density functional is a manner based on the density functional theory.

In the molecular mechanics manner, a molecular force field is generally used for describing impact of various forms of interaction forces on molecular potential energy. It does not compute electronic interaction, and is a simplified model of a molecular structure.

In the deep learning manner, a deep learning model is mainly used for predicting molecular energy, a small data set is used as a training sample set, and the deep learning model is trained based on the training sample set. Therefore, the deep learning model may learn an atomic structure feature, and predict molecular energy based on an extracted feature.

The ab initio method of quantum mechanics has relatively high computation precision, but requires a relatively large computation amount, thereby increasing computation time, and making it difficult to perform large-scale computation by using the ad initio method. The semi-empirical quantum mechanical method of quantum mechanics uses semi-empirical parameters instead of molecular integration, which can accelerate computation, but sacrifices computation precision.

The molecular mechanics force field-based manner can implement a relatively high computation speed, and is also a manner of lowest precision.

In the deep learning manner, an atom feature is not sufficiently utilized, only a few atom types are supported, a capability of capturing molecular long-range interaction is poor, and energy prediction of different molecular conformations is not explored. A pure deep learning manner needs to be improved in terms of precision of molecular energy prediction.

To resolve the foregoing problem, some embodiments provide an artificial intelligence-based molecule processing method and apparatus, an electronic device, a computer-readable storage medium, and a computer program product, which can simultaneously improve precision and increase a speed of molecular energy computation.

The artificial intelligence-based molecule processing method provided in some embodiments may be implemented by a terminal/a server alone; or may be implemented by a terminal in cooperation with a server. For example, the terminal independently undertakes the following artificial intelligence-based molecule processing method. In some embodiments, the terminal sends an energy evaluation request for a target molecule to the server. The server performs the artificial intelligence-based molecule processing method according to the received energy evaluation request for the target molecule, obtains a three-dimensional structure of the target molecule, invokes a neural network model to perform energy error prediction processing on the three-dimensional structure of the target molecule to obtain an energy error of the target molecule, performs first energy computation processing on the three-dimensional structure of the target molecule, to obtain first energy of the target molecule, and performs error correction processing on the first energy of the target molecule based on the energy error of the target molecule, to obtain second energy of the target molecule. Therefore, research and development personnel can perform subsequent analysis and research based on the second energy of the target molecule, for example, determine, by using the second energy of the target molecule, a binding capability of the target molecule to a protein pocket, and screen a candidate drug compound based on the binding capability of the target molecule to the protein pocket.

An electronic device for molecule processing provided in some embodiments may be various types of terminal devices or servers. The server may be an independent physical server, or may be a server cluster or a distributed system formed by a plurality of physical servers, or may be a cloud server that provides a cloud computing service. The terminal may be a smartphone, a tablet computer, a notebook computer, a desktop computer, a smart speaker, a smartwatch, or the like, but is not limited thereto. The terminal and the server may be directly or indirectly connected in a wired or wireless communication manner, which is not limited herein.

A server is used as an example. For example, the server may be a server cluster deployed in a cloud, and AI as a Service (AIaaS) is opened to a user. An AIaaS platform splits several types of common AI services, and provides an independent or packaged service in the cloud. This service mode is similar to an AI theme store, and all users may access, by using an application programming interface, one or more artificial intelligence services provided by using the AIaaS platform.

In some embodiments, an artificial intelligence cloud service may be a molecule processing service, that is, a server in the cloud encapsulates a molecule processing program provided in some embodiments. A user invokes a molecule processing service in a cloud service by using a terminal (running a client, for example, a compound screening client), so that a server deployed in the cloud invokes an encapsulated molecule processing program to obtain a three-dimensional structure of a target molecule, invoke a neural network model to perform energy error prediction processing on the three-dimensional structure of the target molecule to obtain an energy error of the target molecule, perform first energy computation processing on the three-dimensional structure of the target molecule, to obtain first energy of the target molecule, and perform error correction processing on the first energy of the target molecule based on the energy error of the target molecule, to obtain second energy of the target molecule. Therefore, research and development personnel can perform subsequent analysis and research based on the second energy of the target molecule, for example, determine, by using the second energy of the target molecule, a binding capability of the target molecule to a protein pocket, and screen a candidate drug compound based on the binding capability of the target molecule to the protein pocket.

FIG. 2 is a schematic architecture diagram of an artificial intelligence-based molecule processing system according to some embodiments. A terminal 400 connects to a server 200 by using a network 300. The network 300 may be a wide area network or a local area network, or a combination thereof.

The terminal 400 (running a client, such as a compound screening client) may be used for obtaining an energy evaluation request for a target molecule. For example, research and development personnel input the target molecule by using an input interface of the terminal 400, and an energy evaluation request for the target molecule is automatically generated. The terminal 400 sends the energy prediction request for the target molecule to the server 200, and the server 200 obtains a three-dimensional structure of the target molecule, and invokes a neural network model to perform energy error prediction processing on the three-dimensional structure of the target molecule to obtain an energy error of the target molecule; performs first energy computation processing on the three-dimensional structure of the target molecule, to obtain first energy of the target molecule; and performs error correction processing on the first energy of the target molecule based on the energy error of the target molecule, to obtain second energy of the target molecule. The server 200 returns the second energy of the target molecule to the terminal 400. Therefore, research and development personnel can perform subsequent analysis and research based on the second energy of the target molecule, for example, determine, by using the second energy of the target molecule, a binding capability of the target molecule to a protein pocket, and screen a candidate drug compound based on the binding capability of the target molecule to the protein pocket.

In some embodiments, a molecule processing plug-in may be implanted in the client running on the terminal to locally implement the artificial intelligence-based molecule processing method on the client. For example, after obtaining the energy evaluation request for the target molecule, the terminal 400 invokes the molecule processing plug-in, to obtain a three-dimensional structure of the target molecule based on the artificial intelligence-based molecule processing method, invokes a neural network model to perform energy error prediction processing on the three-dimensional structure of the target molecule to obtain an energy error of the target molecule, performs first energy computation processing on the three-dimensional structure of the target molecule, to obtain first energy of the target molecule, and performs error correction processing on the first energy of the target molecule based on the energy error of the target molecule, to obtain second energy of the target molecule. Therefore, research and development personnel can perform subsequent analysis and research based on the second energy of the target molecule, for example, determine, by using the second energy of the target molecule, a binding capability of the target molecule to a protein pocket, and screen a candidate drug compound based on the binding capability of the target molecule to the protein pocket.

In some embodiments, after obtaining the energy evaluation request for the target molecule, the terminal 400 invokes a molecule processing interface (which may be provided as a cloud service, that is, a molecule processing service) of the server 200. The server 200 obtains a three-dimensional structure of a target molecule, invokes a neural network model to perform energy error prediction processing on the three-dimensional structure of the target molecule to obtain an energy error of the target molecule, performs first energy computation processing on the three-dimensional structure of the target molecule, to obtain first energy of the target molecule, and performs error correction processing on the first energy of the target molecule based on the energy error of the target molecule, to obtain second energy of the target molecule. Therefore, research and development personnel can perform subsequent analysis and research based on the second energy of the target molecule, for example, determine, by using the second energy of the target molecule, a binding capability of the target molecule to a protein pocket, and screen a candidate drug compound based on the binding capability of the target molecule to the protein pocket.

FIG. 3 is a schematic structural diagram of an electronic device according to some embodiments. A terminal 400 shown in FIG. 3 includes: at least one processor 410, a memory 450, at least one network interface 420, and a user interface 430. Components in the terminal 400 are coupled together by using a bus system 440. It may be understood that, the bus system 440 is configured to implement connection and communication between the components. In addition to a data bus, the bus system 440 further includes a power bus, a control bus, and a status signal bus. However, for clear description, all types of buses in FIG. 3 are marked as the bus system 440.

The processor 410 may be an integrated circuit chip, and has a signal processing capability, for example, a general-purpose processor, a digital signal processor (DSP), another programmable logic device, a discrete gate or transistor logic device, or a discrete hardware component. The general-purpose processor may be a microprocessor or any conventional processor.

The user interface 430 includes one or more output apparatuses 431 that enable presentation of media content, including one or more speakers and/or one or more visual displays. The user interface 430 further includes one or more input apparatuses 432, including a user interface component that facilitates user input, such as a keyboard, a mouse, a microphone, a touchscreen display, a camera, another input button, and a control.

The memory 450 may be removable, non-removable, or a combination thereof. An exemplary hardware device includes a solid-state memory, a hard disk drive, an optical disk drive, and the like. In some embodiments, the memory 450 may include one or more storage devices that are physically away from the processor 410.

The memory 450 includes a volatile memory or a non-volatile memory, or may include both a volatile memory and a non-volatile memory. The non-volatile memory may be a read-only memory (ROM), and the volatile memory may be a random access memory (RAM). The memory 450 described in some embodiments is intended to include any suitable type of memory.

In some embodiments, the memory 450 can store data to support various operations, and examples of the data include programs, modules, and data structures, or subsets or supersets thereof, as illustrated below.

An operating system 451 includes system programs used for processing various basic system services and executing hardware-related tasks, such as a framework layer, a core library layer, and a driver layer, and is used for implementing various basic services and processing hardware-based tasks.

A network communication module 452 is configured to reach another electronic device by using one or more (wired or wireless) network interfaces 420. An exemplary network interface 420 includes: Bluetooth, wireless compatibility authentication (Wi-Fi), universal serial bus (USB), and the like.

A presentation module 453 is configured to enable presentation of information via one or more output apparatuses 431 (for example, a display and a speaker) associated with the user interface 430 (for example, a user interface for operating a peripheral device and displaying content and information).

An input processing module 454 is configured to detect one or more user inputs or interactions from one of one or more input devices 432 and translate a detected input or interaction.

In some embodiments, an artificial intelligence-based molecule processing apparatus provided in some embodiments may be implemented in a software manner FIG. 3 shows an artificial intelligence-based molecule processing apparatus 455 stored in the memory 450, which may be software in a form of a program, a plug-in, or the like, and includes the following software modules: an obtaining module 4551, a neural network module 4552, a computation module 4553, a correction module 4554, and a training module 4555, which are logical modules. Therefore, any combination or further division may be performed according to an implemented function. Functions of the modules are described below.

As described above, the artificial intelligence-based molecule processing method provided in some embodiments may be implemented by various types of electronic devices.

The following describes the artificial intelligence-based molecule processing method provided in some embodiments. As described above, an electronic device that implements the artificial intelligence-based molecule processing method in some embodiments may be a terminal. Therefore, an execution entity of each operation is not described below.

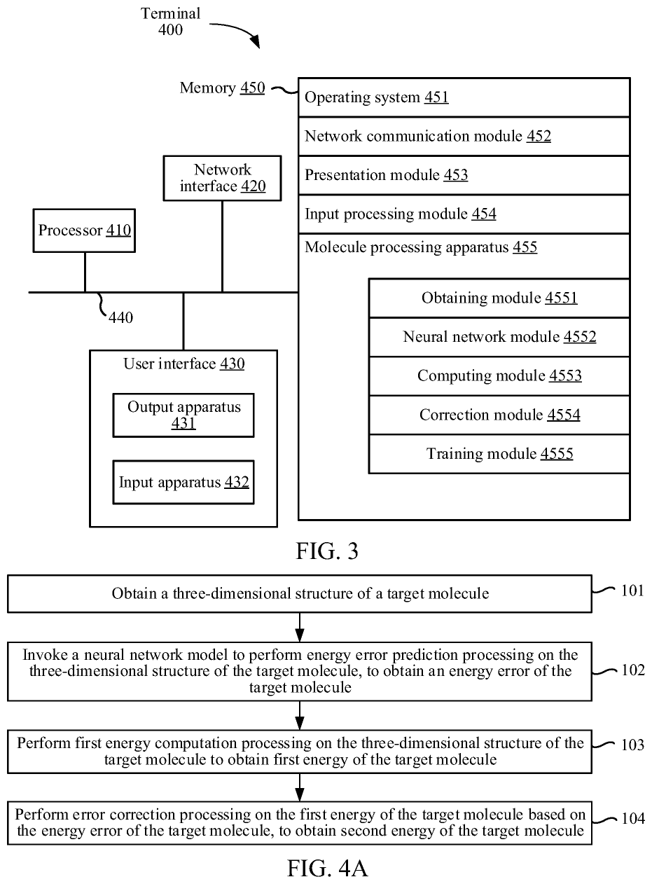

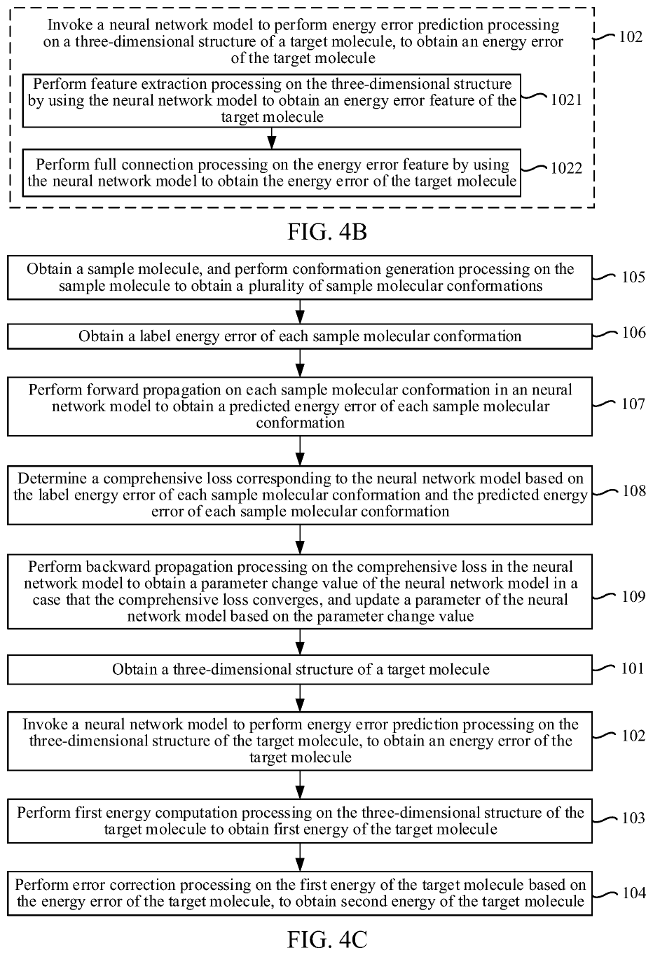

FIG. 4A is a schematic flowchart of an artificial intelligence-based molecule processing method according to some embodiments. References may be made to operations 101 to 104 shown in FIG. 4A for description.

Operation 101: Obtain a three-dimensional structure of a target molecule.

In some embodiments, the target molecule has multiple molecular conformations, and each molecular conformation has a corresponding three-dimensional structure. The target molecule includes at least one atom. For a target molecule having multiple atoms, the three-dimensional structure is a stereoscopic structure formed by multiple atoms and at least one chemical bond.

Operation 102: Invoke a neural network model to perform energy error prediction processing on the three-dimensional structure of the target molecule, to obtain an energy error of the target molecule.

In some embodiments, feature extraction processing is performed on the three-dimensional structure of the target molecule by using the neural network model, to obtain an energy error feature, and the energy error feature is mapped to a difference between computation results obtained in a case that energy computation is performed on the target molecule by using two given energy computation mechanisms. The neural network model performs fitting according to feature mapping to obtain the energy error, and does not really need to perform energy computation on the target molecule by using the two given energy computation mechanisms. This is because a function that can be implemented by the neural network model depends on a training manner, the neural network model is trained by fitting an energy error of a sample molecule, and the energy error of the sample molecule refers to a difference between computation results obtained in a case that energy of the sample molecule is respectively computed according to two energy computation mechanisms. The energy computation mechanism represents a manner of performing energy computation on a given molecule. The manner of energy computation may be energy computation processing based on computational chemistry, and the energy computation processing based on computational chemistry includes a quantum mechanics manner and a molecular mechanics manner. The quantum mechanics manner includes an ab initio method (for example, computation by using a Psi4 tool) and a semi-empirical quantum mechanical method (for example, computation by using a GFN2-xTB program and a GFN-xTB program). The two energy computation mechanisms are respectively corresponding to first energy computation processing and second energy computation processing, precision of the first energy computation processing is less than precision of the second energy computation processing, and a speed of the first energy computation processing is greater than a speed of the second energy computation processing.

Operation 103: Perform the first energy computation processing on the three-dimensional structure of the target molecule to obtain first energy of the target molecule.

In some embodiments, the first energy computation processing and the second energy computation processing may be energy computation processing of computational chemistry, and the energy computation processing of computational chemistry includes a quantum mechanics manner and a molecular mechanics manner. The quantum mechanics manner includes an ab initio method (for example, computation by using a Psi4 tool) and a semi-empirical quantum mechanical method (for example, a GFN2-xTB program and a GFN-xTB program). The first energy computation processing may be the semi-empirical quantum mechanical method, and the second energy computation processing may be the ab initio method. The ab initio method is based on the first principle of quantum chemistry, and uses a rigorous approximation to solve the Schrödinger equation. In the ab initio method, there is a manner based on a wave function and a manner based on density functional. A representative manner based on a wave function is a manner based on the Hartree-Fock Equation, and a representative manner based on density functional is a manner based on the density functional theory.

Operation 104: Perform error correction processing on the first energy of the target molecule based on the energy error of the target molecule, to obtain second energy of the target molecule.

In some embodiments, the error correction processing refers to performing addition or subtraction processing on the first energy and the energy error. When the energy error is used for representing a computation result difference between the first energy computation processing and the second energy computation processing, the error correction processing is performing subtraction processing on the first energy and the energy error to obtain the second energy of the target molecule. When the energy error is used for representing a computation result difference between the second energy computation processing and the first energy computation processing, the error correction processing is performing addition processing on the first energy and the energy error to obtain the second energy of the target molecule.

FIG. 4B is a schematic flowchart of an artificial intelligence-based molecule processing method according to some embodiments. Operation 102 of invoking a neural network model to perform energy error prediction processing on the three-dimensional structure of the target molecule, to obtain an energy error of the target molecule may be implemented by using operation 1021 and operation 1022 shown in FIG. 4B.

Operation 1021: Perform feature extraction processing on the three-dimensional structure by using the neural network model to obtain an energy error feature of the target molecule.

Operation 1022: Perform full connection processing on the energy error feature by using the neural network model to obtain the energy error of the target molecule.

The energy error feature of the three-dimensional structure is extracted in an artificial intelligence manner. A process of performing feature extraction processing in operation 1021 is described in detail in subsequent content, and the energy error of the target molecule is predicted based on the energy error feature, so that a difference between computation results of computing molecular energy of the target molecule by using two types of energy computation processing may be intelligently determined.

In some embodiments, the neural network model includes N cascaded feature networks. Operation 1021 of performing feature extraction processing on the three-dimensional structure by using the neural network model to obtain an energy error feature of the target molecule may be implemented by using the following operations A to C.

Operation A: Perform initial feature extraction processing on each atom in the three-dimensional structure to obtain an initial feature of each atom.

In some embodiments, when a target molecule E includes three atoms (an atom A, an atom B, and an atom C) and one chemical bond (the atom A and the atom B are connected by using the chemical bond), initial feature extraction processing is performed on each atom to obtain an initial attribute feature of each atom in the three-dimensional structure, where the initial attribute feature represents attribute information of the atom, and the attribute information includes at least one of the following: a category of an atom, a property of an atom, and the like. An initial coordinate feature of each atom in the three-dimensional structure is obtained, where the initial coordinate feature represents location information of the atom, the location information refers to three-dimensional coordinates of each atom, and any one of the three atoms is used as an origin to establish a three-dimensional coordinate system, so that three-dimensional coordinates of the other two atoms can be determined, and the following processing is performed for each atom in the three-dimensional structure. For example, the following processing is performed for the atom A: obtaining at least one another atom (that is, the atom B and the atom C) than the atom A in the three-dimensional structure, and obtaining an initial relationship feature between the atom and each of other atoms. The initial relationship feature represents a connection relationship between the atom and the another atom. The initial relationship feature represents a connection relationship between the atom A and the atom B and a connection relationship between the atom A and the atom C. There are two connection relationships: a connected relationship and a not-connected relationship. If two atoms are connected by using the chemical bond, there is a connected relationship between the two atoms. If two atoms are not connected by using the chemical bond, there is a not-connected relationship between the two atoms, and an initial feature of each atom is formed by using the initial attribute feature of the atom, the initial coordinate feature of the atom, and the initial relationship feature of the atom.

The initial attribute feature, the initial relationship feature, and the initial coordinate feature form the initial feature, which can represent not only a category attribute of the atom, but also a relative position of the atom and a connection status by using the chemical bond, thereby effectively improving a representation capability of the initial feature.

Operation B: Perform, in a case that a value of n is 1≤n≤N−1, n^{th} feature extraction processing on an input to an n^{th} feature network in the N cascaded feature networks by using the n^{th} feature network, to obtain an n^{th} feature of each atom, and transmitting the n^{th} feature to an (n+1)th feature network.

In some embodiments, a value range of N meets 2≤N, n is an integer whose value increases from 1, and a value range of n meets 1≤n≤N. In a case that the value of n is 1, the input to the n^{th} feature network is the initial feature of each atom; and in a case that the value of n is 2≤n≤N, the input to the n^{th} feature network is the (n−1)^{th} feature of each atom outputted by an (n−1)^{th} feature network.

In some embodiments, assuming that the value of N is 3, the following processing is performed by still using the atom A as an example: performing first feature extraction processing on the initial feature of the atom A by using a first feature network to obtain a first feature of the atom A, transmitting the first feature to a second feature network to continue to perform second feature extraction processing, and performing second feature extraction processing on the first feature of the atom A by using the second feature network to obtain a second feature of the atom A.

Operation C: Perform, in a case that a value of n is N, attribute feature extraction processing on an (n−1)^{th} feature of each atom by using the n^{th} feature network to obtain an n^{th} attribute feature of each atom, perform coordinate feature extraction processing on the (n−1)^{th} feature of each atom by using the n^{th} feature network to obtain an n^{th} coordinate feature of each atom, and form the energy error feature by using the n^{th} attribute feature and the n^{th} coordinate feature of each atom.

In some embodiments, assuming that the value of N is 3, the following processing is performed by still using the atom A as an example: performing third attribute feature extraction processing on the second feature of the atom A by using a third feature network, to obtain a third attribute feature of the atom A; performing third coordinate feature extraction processing on the second feature of the atom A by using the third feature network, to obtain a third coordinate feature of the atom A; and using the third attribute feature of the atom A and the third coordinate feature of the atom A as an energy error feature.

In some embodiments, the coordinate feature of each atom and the attribute feature of each atom can be obtained in an iterative manner, and then coordinate features of multiple atoms and attribute features of the multiple atoms form an energy error feature, which can effectively improve a feature expression capability of the energy error feature, thereby improving precision of a subsequently predicted energy error.

In some embodiments, operation B of performing n^{th} feature extraction processing on an input to an n^{th} feature network in the N cascaded feature networks by using the n^{th} feature network, to obtain an n^{th} feature of each atom may be implemented by performing the following operations B1 to B3 on each atom by using the n^{th} feature network (the atom A in the target molecule E is used as an example for description).

Operation B1: Obtain other atoms than the atom in the three-dimensional structure. Still using the target molecule E as an example, the other atoms are another atom B and another atom C than the atom A.

Operation B2: Perform first mapping processing on the (n−1)^{th} feature of the atom and an (n−1)^{th} feature of each of the other atoms, to obtain an n^{th} association feature of the atom corresponding to each of the other atoms.

Extract an (n−1)^{th} coordinate feature of the atom from the (n−1)^{th} feature of the atom, and extract an (n−1)^{th} coordinate feature of each of the other atoms from the (n−1)^{th} feature of each of the other atoms; extract an (n−1)^{th} attribute feature of the atom from the (n−1)^{th} feature of the atom, and extract an (n−1)^{th} attribute feature of each of the other atoms from the (n−1)^{th} feature of each of the other atoms; and extract an (n−1)^{th} relationship feature of the atom from the (n−1)^{th} feature of the atom; and The following processing is performed for each of the other atoms (hereinafter described by using the atom B as an example): extracting an (n−1)^{th} relationship feature of the atom for the another atom from the (n−1)^{th} relationship feature of the atom; obtaining a first feature distance between the (n−1)^{th} coordinate feature of the atom and the (n−1)^{th} coordinate feature of the another atom; and performing first fusion processing on a square of the first feature distance, the (n−1)^{th} attribute feature of the atom, the (n−1)^{th} attribute feature of the another atom, and the (n−1)^{th} relationship feature of the atom for the another atom, to obtain the n^{th} association feature of the atom corresponding to the another atom.

In some embodiments, a feature distance between any two atoms and a connection relationship between any two atoms are considered in an association feature, so that the association feature can learn global information of a three-dimensional structure, and a global information learning capability of a neural network model can be improved.

In some embodiments, it is assumed that the value of N is 3, and the atom A and the another atom B are still used as examples for description. A second coordinate feature of the atom A is extracted from the second feature of the atom A, a second coordinate feature of the atom B is extracted from a second feature of the atom B, a second attribute feature of the atom A is extracted from the second feature of the atom A, and a second attribute feature of the atom B is extracted from the second feature of the atom B. The (n−1)^{th} relationship feature is an initial relationship feature, that is, the relationship feature does not change due to a feature iteration, a second relationship feature of the atom A (an initial relationship feature of the atom A) is extracted from the second feature of the atom A, and a second relationship feature of the atom B (an initial relationship feature of the atom B) is extracted from the second feature of the atom B.

In some embodiments, for performing subsequent processing on the another atom B, refer to formula (1):

$${m}_{ij} = {ϕ}_{e} ( {h}_{i}^{n - 1} , {h}_{j}^{n - 1} , {‖ {x}_{i}^{n - 1} - {x}_{j}^{n - 1} ‖}^{2} , {a}_{ij} ) , \qquad ( 1 )$$

where hᵢⁿ⁻¹ is the (n−1)^{th} attribute feature of the atom A, hⱼⁿ⁻¹ is the (n−1)^{th} attribute feature of the atom B, xᵢⁿ⁻¹ is the (n−1)^{th} coordinate feature of the atom A, xⱼⁿ⁻¹ is the (n−1)^{th} coordinate feature of the atom B, aᵢⱼ is the (n−1)^{th} relationship feature of the atom A corresponding to the atom B (that is, the initial relationship feature of the atom A corresponding to the atom B), ϕₑ represents first fusion processing, ∥xᵢⁿ⁻¹−xⱼⁿ⁻¹∥ represents a second feature distance between the (n−1)^{th} coordinate feature of the atom A and the (n−1)^{th} coordinate feature of the atom B, and mᵢⱼ is an n^{th} association feature of the atom A corresponding to the another atom B.

Operation B3: Perform second mapping processing on the (n−1)^{th} feature of the atom and the n^{th} association feature of the atom corresponding to each of the other atoms, to obtain the n^{th} feature of the atom.

Perform summation processing on n^{th} association features of the atom corresponding to multiple other atoms, to obtain an n^{th} association feature of the atom; perform second fusion processing on the (n−1)^{th} attribute feature of the atom and the n^{th} association feature of the atom, to obtain the n^{th} attribute feature of the atom; obtain a first feature difference between the (n−1)^{th} coordinate feature of the atom and the (n−1)^{th} coordinate feature of each of the other atoms; perform linear mapping processing on the n^{th} association feature of the atom corresponding to each of the other atoms, to obtain a weight of each of the other atoms; perform weighted average processing on first feature differences of multiple other atoms based on the weight of each of the other atoms, to obtain a weighted average result corresponding to the atom; perform summation processing on the weighted average result of the atom and the (n−1)^{th} coordinate feature of the atom to obtain the n^{th} coordinate feature of the atom; use an initial relationship feature of the atom as an n^{th} relationship feature of the atom; and forming the n^{th} feature of the atom by using the n^{th} relationship feature of the atom, the n^{th} attribute feature of the atom, and the n^{th} coordinate feature of the atom.

In some embodiments, a feature distance between any two atoms and a connection relationship between any two atoms are considered in an n^{th} feature, so that the n^{th} feature can learn global information of a three-dimensional structure, and a global information learning capability of a neural network model can be improved.

In some embodiments, summation processing is performed on n^{th} association features of the atom A corresponding to multiple other atoms (the another atom B and the another atom C), to obtain the n^{th} association feature of the atom A. Refer to formula (2):

$${m}_{i} = {\sum }_{j ∈ ( i )} {m}_{ij} , \qquad ( 2 )$$

where i is the atom A, j is the atom B and the atom C, mᵢ is the n^{th} association feature of the atom A, and mᵢⱼ is an n^{th} association feature of the atom A corresponding to the atom B and an n^{th} association feature of the atom A corresponding to the atom C.

In some embodiments, second fusion processing is performed on the (n−1)^{th} attribute feature of the atom A and the n^{th} association feature of the atom A, to obtain the n^{th} attribute feature of the atom A. Refer to formula (3):

$${h}_{i}^{n} = {ϕ}_{h} ( {h}_{i}^{n - 1} , {m}_{i} ) , \qquad ( 3 )$$

where i is the atom A, hᵢⁿ is the n^{th} attribute feature of the atom A, hᵢⁿ⁻¹ is the (n−1)^{th} attribute feature of the atom A, mᵢ is the n^{th} association feature of the atom A, and ϕₕ is second fusion processing.

In some embodiments, a first feature difference between the (n−1)^{th} coordinate feature of the atom A and the (n−1)^{th} coordinate feature of another atom (the another atom B and the another atom C) is obtained. Weighted average processing is performed on first feature differences of multiple other atoms (the another atom B and the another atom C) by using a linear mapping result of the n^{th} association feature of the atom A corresponding to each of other atoms (the another atom B and the another atom C) as a weight, to obtain a weighted average result corresponding to the atom A. Summation processing is performed on the weighted average result of the atom A and the (n−1)^{th} coordinate feature of the atom A to obtain the n^{th} coordinate feature of the atom A. For the foregoing processing on the atom A and the other atoms, refer to formula (4).

$${xi}_{n}^{} = {x}_{i}^{n - 1} + M {\sum }_{j \neq i} ( {x}_{i}^{n - 1} - {x}_{j}^{n - 1} ) {φ}_{x} ( {m}_{ij} ) , \qquad ( 4 )$$

where xᵢⁿ is the n^{th} coordinate feature of the atom A, xᵢⁿ⁻¹ is the (n−1)^{th} coordinate feature of the atom A, xⱼⁿ⁻¹ is the (n−1)^{th} coordinate feature of the another atom, m u is the n^{th} association feature of the atom A corresponding to the another atom B, M is a reciprocal of a quantity of atoms in the target molecule, and φₓ is coordinate linear mapping processing.

In some embodiments, the initial relationship feature of the atom A is used as the n^{th} relationship feature of the atom A. The n^{th} feature of the atom is formed by using the n^{th} relationship feature aᵢⱼ of the atom A corresponding to each of the other atoms, the n^{th} attribute feature hᵢⁿ of the atom A, and the n^{th} coordinate feature of the atom A.

In some embodiments, operation C of performing attribute feature extraction processing on an (n−1)^{th} feature of each atom by using the n^{th} feature network to obtain an n^{th} attribute feature of each atom may be implemented by using the following technical solution: extracting, from the (n−1)^{th} feature of each atom, an (n−1)^{th} coordinate feature of the atom, an (n−1)^{th} attribute feature of the atom, and an (n−1)^{th} relationship feature of the atom; and performing the following processing for each atom: obtaining other atoms than the atom in the three-dimensional structure, and performing the following processing for each of the other atoms: extracting an (n−1)^{th} relationship feature of the atom for the another atom from the (n−1)^{th} relationship feature of the atom, and obtaining a second feature distance between the (n−1)^{th} coordinate feature of the atom and an (n−1)^{th} coordinate feature of the another atom; performing first fusion processing on a square of the second feature distance, the (n−1)^{th} attribute feature of the atom, the (n−1)^{th} attribute feature of the another atom, and the (n−1)^{th} relationship feature of the atom for the another atom, to obtain an n^{th} association feature of the atom corresponding to the another atom; performing summation processing on n^{th} association features of the atom corresponding to multiple other atoms, to obtain an n^{th} association feature of the atom; and performing second fusion processing on the (n−1)^{th} attribute feature of the atom and the n^{th} association feature of the atom, to obtain the n^{th} attribute feature of the atom.

In some embodiments, from the (n−1)^{th} feature of each atom, the (n−1)^{th} coordinate feature of the atom, the (n−1)^{th} attribute feature of the atom, and the (n−1)^{th} relationship feature of the atom are extracted. In some embodiments, assuming that the value of N is 3, the following processing is performed by still using the atom A, the atom B, and the atom C as an example: extracting a second coordinate feature of the atom A from the second feature of the atom A, extracting a second coordinate feature of the atom B from the second feature of the atom B, extracting a second coordinate feature of the atom C from the second feature of the atom C, extracting a second attribute feature of the atom A from the second feature of the atom A, extracting a second attribute feature of the atom B from the second feature of the atom B, and extracting a second attribute feature of the atom C from the second feature of the atom C. The (n−1)^{th} relationship feature is an initial relationship feature, that is, the relationship feature does not change due to a feature iteration. The second relationship feature of the atom A (the initial relationship feature of the atom A) is extracted from the second feature of the atom A, the second relationship feature of the atom B (the initial relationship feature of the atom B) is extracted from the second feature of the atom B, and the second relationship feature of the atom C (the initial relationship feature of the atom C) is extracted from the second feature of the atom C.

In some embodiments, for each atom (the atom A is used as an example for description), the another atom B and the another atom C than the atom in the three-dimensional structure are obtained, and subsequent processing (the another atom B is used as an example for description) is performed for each of the other atoms. Refer to formula (5):

$${m}_{ij} = {ϕ}_{e} ( {h}_{i}^{n - 1} , {h}_{j}^{n - 1} , {‖ {x}_{i}^{n - 1} - {x}_{j}^{n - 1} ‖}^{2} , {a}_{ij} ) , \qquad ( 5 )$$

where hᵢⁿ is the (n−1)^{th} attribute feature of the atom A, h; is the (n−1)^{th} attribute feature of the atom B, xᵢⁿ is the (n−1)^{th} coordinate feature of the atom A, x; is the (n−1)^{th} coordinate feature of the atom B, aᵢⱼ is the (n−1)^{th} relationship feature of the atom A corresponding to the atom B (that is, the initial relationship feature of the atom A corresponding to the atom B), ϕ_{c} represents first fusion processing, ∥xᵢⁿ−xⱼⁿ∥ represents a second feature distance between the (n−1)^{th} coordinate feature of the atom A and the (n−1)^{th} coordinate feature of the atom B, and m u is an n^{th} association feature of the atom A corresponding to the another atom B.

In some embodiments, summation processing is performed on n^{th} association features of the atom A corresponding to multiple other atoms (the another atom B and the another atom C), to obtain the n^{th} association feature of the atom A. Refer to formula (6):

$${m}_{i} = {\sum }_{j ∈ ( i )} {m}_{ij} , \qquad ( 6 )$$

where i is the atom A, j is the atom B and the atom C, mᵢ is the n^{th} association feature of the atom A, and is mᵢⱼ the n^{th} association feature of the atom A corresponding to the another atom B.

In some embodiments, second fusion processing is performed on the (n−1)^{th} attribute feature of the atom A and the n^{th} association feature of the atom A, to obtain the n^{th} attribute feature of the atom A. Refer to formula (7):

$${h}_{i}^{n} = {ϕ}_{h} ( {h}_{i}^{n - 1} , {m}_{i} ) , \qquad ( 7 )$$

where i is the atom A, hᵢⁿ⁻¹ is the n^{th} attribute feature of the atom A, hᵢⁿ is the (n−1)^{th} attribute feature of the atom A, mᵢ is the n^{th} association feature of the atom A, and ϕₕ is second fusion processing.

For a specific implementation of operation C of performing coordinate feature extraction processing on the (n−1)^{th} feature of each atom by using the n^{th} feature network to obtain an n^{th} coordinate feature of each atom, refer to a specific implementation of obtaining the (n−1)^{th} coordinate feature.

In some embodiments, referring to FIG. 4C, before energy error prediction processing is performed on the three-dimensional structure of the target molecule by invoking the neural network model to obtain the energy error of the target molecule, operation 105 to operation 109 shown in FIG. 4C may be further performed.

Operation 105: Obtain a sample molecule, and perform conformation generation processing on the sample molecule to obtain multiple sample molecular conformations.

In some embodiments, the sample molecule comes from any molecule data set, for example, a QMugs data set, and conformation generation processing may be performed on the sample molecule to obtain multiple sample molecular conformations. The conformation generation processing includes twisting a chemical bond. The obtained multiple sample molecular conformations include a molecular conformation of the sample molecule itself and another molecular conformation obtained by twisting the chemical bond. The conformation generation processing cannot change a type of each atom in the molecule, but may change a coordinate of each atom in the molecule and a distance between atoms.

Operation 106: Obtain a label energy error of each sample molecular conformation.

In some embodiments, operation 106 of obtaining a label energy error of each sample molecular conformation may be implemented by using the following technical solution: performing the following processing for each sample molecular conformation: performing first energy computation processing on the sample molecular conformation to obtain first energy of the sample molecular conformation; performing second energy computation processing on the sample molecular conformation to obtain second energy of the sample molecular conformation; and obtaining a first difference between the second energy of the sample molecular conformation and the first energy of the sample molecular conformation as the label energy error of the sample molecular conformation.

In some embodiments, a high-precision second energy processing manner is used for computing molecular energy of a molecule. For example, a DFT computation theory hierarchy and a base group “WB97X-D3/def2-TZVP” are used for computing quantum mechanics energy (single-point energy) of the molecule as molecular energy, and the computed molecular energy is denoted as E_dft. In addition, a high-speed first energy processing manner is used for computing the molecular energy of the molecule. For example, a semi-empirical quantum mechanical method is used for computing semi-empirical quantum mechanical energy of the molecule as the molecular energy, and the computed molecular energy is denoted as E_xtb. A difference E_delta=E_dft−E_xtb between the two is denoted as a label energy error (Label) of a molecular conformation.

Operation 107: Perform forward propagation on each sample molecular conformation in an initialized neural network model to obtain a predicted energy error of each sample molecular conformation.

In some embodiments, feature extraction processing is performed on the three-dimensional structure by using the initialized neural network model, to obtain an energy error feature of the target molecule; and full connection processing is performed on the energy error feature by using the initialized neural network model, to obtain an energy error of the target molecule. For a processing process of the initialized neural network model, references may be made to the foregoing embodiment. A difference lies only in that a parameter used in the processing process is an initialized parameter, not a parameter obtained after training.

Operation 108: Determine a comprehensive loss corresponding to the neural network model based on the label energy error of each sample molecular conformation and the predicted energy error of each sample molecular conformation.

In some embodiments, operation 108 of determining a comprehensive loss corresponding to the neural network model based on the label energy error of each sample molecular conformation and the predicted energy error of each sample molecular conformation may be implemented by using the following technical solution: performing the following processing for each sample molecular conformation: determining a first root mean square error of the sample molecular conformation based on the label energy error of the sample molecular conformation and the predicted energy error of the sample molecular conformation; and obtaining other sample molecular conformations of the sample molecule than the sample molecular conformation; and performing the following processing for each of the other sample molecular conformations: determining a second difference between the label energy error of the sample molecular conformation and a label energy error of the another sample molecular conformation, and determining a third difference between the predicted energy error of the sample molecular conformation and a predicted energy error of the another sample molecular conformation; performing root mean square processing on the second difference and the third difference to obtain a second root mean square error of the sample molecular conformation corresponding to the another sample molecular conformation; performing summation processing on second root mean square errors of the sample molecular conformation corresponding to multiple other sample molecular conformations, to obtain a third root mean square error of the sample molecular conformation; and performing third fusion processing on first root mean square errors of multiple sample molecular conformations and third root mean square errors of the multiple sample molecular conformations to obtain a comprehensive loss corresponding to the neural network model.

In some embodiments, for the comprehensive loss of the neural network model, refer to formula (8):

$$( \hat{E} , E ) = {\sum }_{i} {L}_{2} ( {\hat{E}}_{i} , {E}_{i} ) + α {\sum }_{i} {L}_{2} ( {\hat{E}}_{i} - {\hat{E}}_{m ( i )} , {E}_{i} - {E}_{m ( i )} ) , \qquad ( 8 )$$

where L₂(Êᵢ,Eᵢ) is the first root mean square error, E is a label energy error of a sample molecular conformation i, Eᵢ is a predicted energy error of the sample molecular conformation i, L₂ represents root mean square processing, Êᵢ−Êₘ₍ᵢ₎ is a second difference between the label energy error of the sample molecular conformation i and a label energy error of another sample molecular conformation m(i), Eᵢ−Eₘ₍ᵢ₎ is a third difference between the predicted energy error of the sample molecular conformation i and a predicted energy error of the another sample molecular conformation m(i), L₂(Êᵢ−Êₘ₍ᵢ₎−Eₘ₍ᵢ₎) is a third root mean square error of the sample molecular conformation i, and (Ê, E) is the comprehensive loss.

Operation 109: Perform backward propagation processing on the comprehensive loss in the neural network model to obtain a parameter change value of the neural network model in a case that the comprehensive loss converges, and update a parameter of the neural network model based on the parameter change value.

In some embodiments, backward propagation may be implemented by using a backward propagation algorithm. The backward propagation algorithm is mainly iterated repeatedly by using two phases (excitation propagation and weight update) until a response of a network to an input reaches a predetermined target range. A learning process of the backward propagation algorithm includes a forward propagation process and a backward propagation process. In the forward propagation process, input information is processed layer by layer through an input layer and then a hidden layer, and then transmitted to an output layer. If an expected output value is not obtained at the output layer, a square sum of an error between the output value and the expected value is used as a target function, which is transferred to the backward propagation. A partial derivative of the target function to each neuron weight value is obtained layer by layer, to form a gradient of the target function to a weight vector, which is used as a basis for weight value modification. Network learning is completed in a weight value modification process. When the comprehensive loss converges to the expected value, learning of the neural network model ends.

The following describes an exemplary application of the embodiments in an actual application scenario.

A terminal (running a client, such as a compound screening client) may be used for obtaining an energy evaluation request for a target molecule. For example, research and development personnel input the target molecule by using an input interface of the terminal, and an energy evaluation request for the target molecule is automatically generated. The terminal sends the energy prediction request for the target molecule to a server, and the server obtains a three-dimensional structure of the target molecule, and invokes a neural network model to perform energy error prediction processing on the three-dimensional structure of the target molecule to obtain an energy error of the target molecule; performs first energy computation processing on the three-dimensional structure of the target molecule, to obtain first energy of the target molecule; and performs error correction processing on the first energy of the target molecule based on the energy error of the target molecule, to obtain second energy of the target molecule. The server returns the second energy of the target molecule to the terminal. Therefore, research and development personnel can perform subsequent analysis and research based on the second energy of the target molecule, for example, determine, by using the second energy of the target molecule, a binding capability of the target molecule to a protein pocket, and screen a candidate drug compound based on the binding capability of the target molecule to the protein pocket.

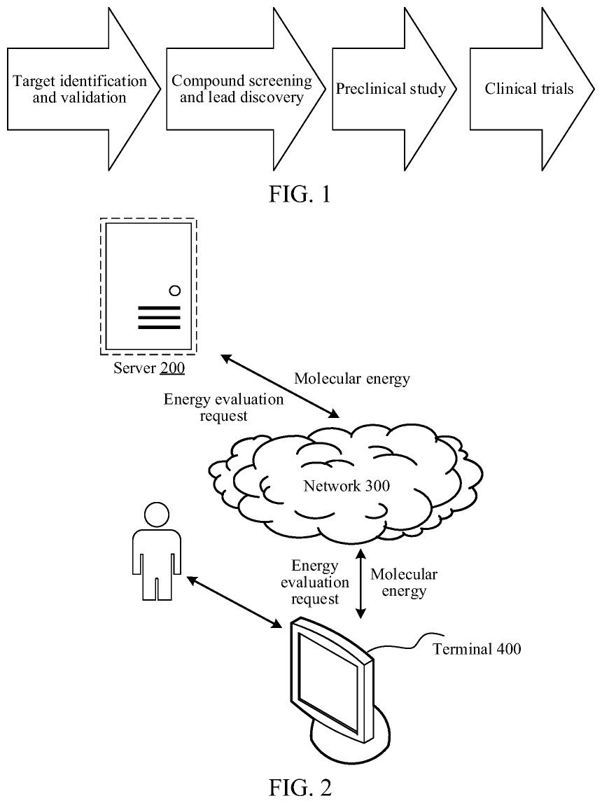

The artificial intelligence-based molecule processing method provided in some embodiments may be used in a new drug research and development process and a material development process. The new drug research and development process is used as an example. As shown in FIG. 1, in the new drug research and development process, after target identification and validation are completed, a candidate drug compound needs to be screened. In a screening process, energy of a molecule generally needs to be computed in a scenario such as computation of molecular properties and computation of a binding capability of a molecule to a protein pocket. A computation result is helpful for drug research and development personnel to analyze molecular properties, binding capabilities of molecules and protein pockets, and the like and helps research and development personnel to design more effective drug molecules, greatly improving research and development efficiency and reducing drug research and development costs.

First, a training data set of a deep quantum chemistry model (DeepQC) needs to be constructed. An enhanced data set is constructed based on any data set (for example, a QMugs data set). There are 660,000 types of molecules in the enhanced data set, and each molecule has three molecular conformations. Therefore, there are nearly 2 million pieces of data in total.

For each molecular conformation in the enhanced data set, a high-precision second energy processing manner is used for computing molecular energy of a molecule. For example, a DFT computation theory hierarchy and a base group “WB97X-D3/def2-TZVP” are used for computing quantum mechanics energy (single-point energy) of the molecule as molecular energy, and the computed molecular energy is denoted as E_dft. In addition, a high-speed first energy processing manner is used for computing the molecular energy of the molecule. For example, a semi-empirical quantum mechanical method is used for computing semi-empirical quantum mechanical energy of the molecule as the molecular energy, and the computed molecular energy is denoted as E_xtb. A difference E_delta=E_dft−E_xtb between the two is denoted as a label energy error (Label) of a molecular conformation. Quantum mechanics computation related to the second energy processing manner is implemented by using a Psi4 tool, and the semi-empirical quantum mechanical computation related to the first energy processing manner is implemented by using an xTB tool. The enhanced data set is divided into a training set, a validation set, and a test set of DeepQC according to a ratio of 8:1:1.

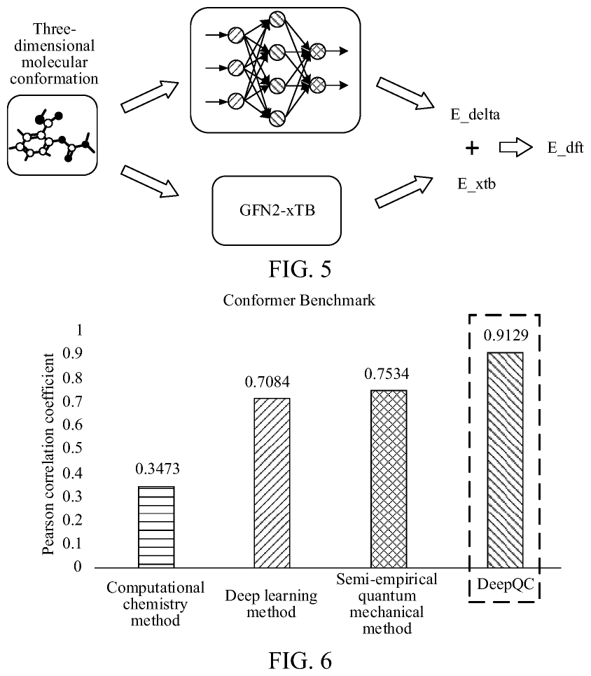

An architecture of the DeepQC model is shown in FIG. 5. The DeepQC model includes a neural network model involving deep learning and a first energy processing manner involving computational chemistry. The neural network model may be an equivariant graph neural network. An input to the neural network model is a three-dimensional structure coordinate of a molecule, and an output of the neural network model is an E_delta predicted value. In a training process, a comprehensive loss is computed between an E_delta predicted value obtained through forward propagation and an E_delta label value, backward propagation is performed on the comprehensive loss to obtain a gradient of each network layer, and a parameter of the neural network model is updated by using an adaptive moment estimation algorithm. For the comprehensive loss of the neural network model, refer to formula (9):

$$( \hat{E} , E ) = {\sum }_{i} {L}_{2} ( {\hat{E}}_{i} , {E}_{i} ) + α {\sum }_{i} {L}_{2} ( {\hat{E}}_{i} - {\hat{E}}_{m ( i )} , {E}_{i} - {E}_{m ( i )} ) . \qquad ( 9 )$$

The loss function includes two parts. The first part is a root mean square error between the E_delta predicted value and the E_delta label value as outputted by the model. The second part is a root mean square error between a prediction difference and a label difference. The prediction difference refers to an E_delta label value difference between different conformations of the same molecule. The label difference refers to an E_delta label value difference between different conformations of the same molecule. L₂ (Êᵢ,Eᵢ) is the first root mean square error, Êᵢ is a label energy error of a sample molecular conformation i, Eᵢ is a predicted energy error of the sample molecular conformation i, L₂ represents root mean square processing, Êᵢ−Eₘ₍ᵢ₎ is a second difference between the label energy error of the sample molecular conformation i and a label energy error of another sample molecular conformation m(i), Eᵢ−Eₘ₍ᵢ₎ is a third difference between the predicted energy error of the sample molecular conformation i and a predicted energy error of the another sample molecular conformation m(i), L₂(Êᵢ−Êₘ₍ᵢ₎,Eᵢ−Eₘ₍ᵢ₎) is a third root mean square error of the sample molecular conformation i, and (Ê, E) is the comprehensive loss.

Training hyper-parameters of the DeepQC model are shown in Table 1:

TABLE 1
Hyper-parameter table of the DeepQC model

| | |
|---|---|
| Quantity of layers | 8 |
| Learning rate | 0.002 |
| Training set size | 32 |
| Weight attenuation | 0.000001 |
| Training repeats | 100 |
| Discard rate | 0.1 |

Energy of a molecule may be predicted by using a trained DeepQC model, and a three-dimensional coordinate of the molecule is inputted into the DeepQC model. First energy E_xtb may be obtained by using the first energy computation processing (for example, processing by using a GFN2-xTB program), and an energy error E_delta may be predicted by using the neural network model (for example, an equivariant graph neural network), and a final predicted energy value E_dft is obtained by accumulating the two.

Energy of a molecule needs to be computed in a new drug/new material research and development process. In a computation method of a related technology, a quantum mechanical method has a large computation amount and computation time consumption, and cannot perform large-scale computation. Prediction of a deep learning method in a long range is poor, and precision needs to be improved.

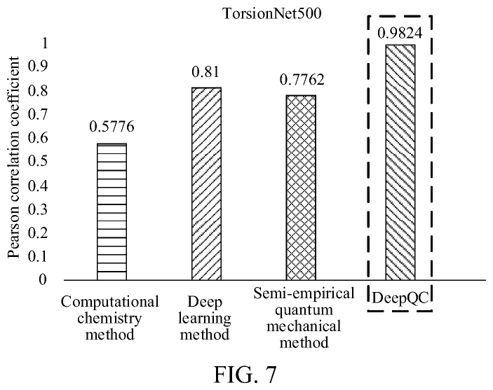

The DeepQC model provided in some embodiments aims to maintain relatively high precision while a computation amount is small. To better validate an effect of the DeepQC model, the DeepQC model is validated on a test set, tested on two data sets, Conformer Benchmark and TorsionNet 500, for computational precision, and compared to the computational chemistry method, the semi-empirical quantum mechanical method, and the deep learning method. A vertical axis of FIG. 6 indicates a correlation between molecular energy computed by using various methods in Conformer Benchmark and a theoretical value, and a vertical axis of FIG. 7 indicates a correlation between molecular energy computed by using various methods in TorsionNet 500 and the theoretical value, where the theoretical value is molecular energy obtained through computation by using a high-precision quantum mechanics method. In terms of an algorithm speed, the DeepQC model provided in some embodiments is hundreds of times faster than a high-precision quantum mechanics method.

The following continues to describe an example structure when the artificial intelligence-based molecule processing apparatus 455 provided in some embodiments is implemented as a software module. In some embodiments, as shown in FIG. 3, software modules in the artificial intelligence-based molecule processing apparatus 455 as stored in the memory 450 may include: an obtaining module 4551, configured to obtain a three-dimensional structure of a target molecule; a neural network module 4552, configured to invoke a neural network model to perform energy error prediction processing on the three-dimensional structure of the target molecule, to obtain an energy error of the target molecule; the neural network model being trained by fitting an energy error of a sample molecule, the energy error of the sample molecule referring to a difference between computation results obtained in a case that energy of the sample molecule is separately computed according to two energy computation mechanisms, the two energy computation mechanisms including first energy computation processing and second energy computation processing, precision of the first energy computation processing being less than precision of the second energy computation processing, and a speed of the first energy computation processing being greater than a speed of the second energy computation processing; a computation module 4553, configured to perform the first energy computation processing on the three-dimensional structure of the target molecule to obtain first energy of the target molecule; and a correction module 4554, configured to perform error correction processing on the first energy of the target molecule based on the energy error of the target molecule, to obtain second energy of the target molecule.

In some embodiments, the neural network module 4552 is further configured to: perform feature extraction processing on the three-dimensional structure by using the neural network model to obtain an energy error feature of the target molecule; and perform full connection processing on the energy error feature by using the neural network model to obtain the energy error of the target molecule.

In some embodiments, the neural network model includes N cascaded feature networks, and the neural network module 4552 is further configured to: perform initial feature extraction processing on each atom in the three-dimensional structure to obtain an initial feature of each atom; and perform, in a case that a value of n is 1≤n≤N−1, n^{th} feature extraction processing on an input to an n^{th} feature network in the N cascaded feature networks by using the n^{th} feature network, to obtain an n^{th} feature of each atom, and transmitting the n^{th} feature to an (n+1)^{th} feature network; or perform, in a case that a value of n is N, attribute feature extraction processing on an (n−1)^{th} feature of each atom by using the n^{th} feature network to obtain an n^{th} attribute feature of each atom, perform coordinate feature extraction processing on the (n−1)^{th} feature of each atom by using the n^{th} feature network to obtain an n^{th} coordinate feature of each atom, and form the energy error feature by using the n^{th} attribute feature and the n^{th} coordinate feature of each atom; a value range of N meeting 2≤N, n being an integer whose value increases from 1, and a value range of n meeting 1≤n≤N−1; and in a case that the value of n is 1, the input to the n^{th} feature network being the initial feature of each atom; and in a case that the value of n is 2≤n≤N, the input to the n^{th} feature network being the (n−1)^{th} feature of each atom outputted by an (n−1)^{th} feature network.

In some embodiments, the neural network module 4552 is further configured to: obtain an initial attribute feature of each atom in the three-dimensional structure, and obtain an initial coordinate feature of each atom in the three-dimensional structure; the initial attribute feature representing attribute information of the atom, and the initial coordinate feature representing location information of the atom; and perform the following processing for each atom of the three-dimensional structure: obtain at least one other atom than the atom in the three-dimensional structure, and obtain an initial relationship feature between the atom and each of the other atoms, the initial relationship feature representing a connection relationship between the atom and another atom; and form the initial feature of each atom by using the initial attribute feature of the atom, the initial coordinate feature of the atom, and the initial relationship feature of the atom.

In some embodiments, the neural network module 4552 is further configured to: perform the following processing on each atom by using the n^{th} feature network: obtaining other atoms than the atom in the three-dimensional structure; performing first mapping processing on the (n−1)^{th} feature of the atom and an (n−1)^{th} feature of each of the other atoms, to obtain an n^{th} association feature of the atom corresponding to each of the other atoms; and performing second mapping processing on the (n−1)^{th} feature of the atom and the n^{th} association feature of the atom corresponding to each of the other atoms, to obtain the n^{th} feature of the atom.

In some embodiments, the neural network module 4552 is further configured to: extract an (n−1)^{th} coordinate feature of the atom from the (n−1)^{th} feature of the atom, and extract an (n−1)^{th} coordinate feature of each of the other atoms from the (n−1)^{th} feature of each of the other atoms; extract an (n−1)^{th} attribute feature of the atom from the (n−1)^{th} feature of the atom, and extract an (n−1)^{th} attribute feature of each of the other atoms from the (n−1)^{th} feature of each of the other atoms; and extract an (n−1)^{th} relationship feature of the atom from the (n−1)^{th} feature of the atom; and perform the following processing for each of the other atoms: extracting an (n−1)^{th} relationship feature of the atom for the another atom from the (n−1)^{th} relationship feature of the atom; obtaining a first feature distance between the (n−1)^{th} coordinate feature of the atom and the (n−1)^{th} coordinate feature of the another atom; and performing first fusion processing on a square of the first feature distance, the (n−1)^{th} attribute feature of the atom, the (n−1)^{th} attribute feature of the another atom, and the (n−1)^{th} relationship feature of the atom for the another atom, to obtain the n^{th} association feature of the atom corresponding to the another atom.

In some embodiments, the neural network module 4552 is further configured to: perform, in a case that a quantity of other atoms is multiple, summation processing on n^{th} association features of the atom corresponding to multiple other atoms, to obtain an n^{th} association feature of the atom; and perform second fusion processing on the (n−1)^{th} attribute feature and the n^{th} association feature of the atom, to obtain the n^{th} attribute feature of the atom; obtain a first feature difference between the (n−1)^{th} coordinate feature of the atom and the (n−1)^{th} coordinate feature of each of the other atoms; perform weighted average processing on first feature differences of multiple other atoms by using an n^{th} association feature of the atom corresponding to each of the other atoms as a weight, to obtain a weighted average result corresponding to the atom; perform summation processing on the weighted average result of the atom and the (n−1)^{th} coordinate feature of the atom to obtain the n^{th} coordinate feature of the atom; use an initial relationship feature of the atom as an n^{th} relationship feature of the atom; and form the n^{th} feature of the atom by using the n^{th} relationship feature of the atom, the n^{th} attribute feature of the atom, and the n^{th} coordinate feature of the atom.

In some embodiments, the neural network module 4552 is further configured to: extract an (n−1)^{th} coordinate feature of the atom from the (n−1)^{th} feature of the atom, and extract an (n−1)^{th} coordinate feature of each of the other atoms from the (n−1)^{th} feature of each of the other atoms; extract an (n−1)^{th} attribute feature of the atom from the (n−1)^{th} feature of the atom, and extract an (n−1)^{th} attribute feature of each of the other atoms from the (n−1)^{th} feature of each of the other atoms; and extract an (n−1)^{th} relationship feature of the atom from the (n−1)^{th} feature of the atom; and perform the following processing for each of the other atoms: extract an (n−1)^{th} relationship feature of the atom for the another atom from the (n−1)^{th} relationship feature of the atom; obtain a second feature distance between the (n−1)^{th} coordinate feature of the atom and the (n−1)^{th} coordinate feature of the another atom; and perform first fusion processing on a square of the second feature distance, the (n−1)^{th} attribute feature of the atom, the (n−1)^{th} attribute feature of the another atom, and the (n−1)^{th} relationship feature of the atom for the another atom, to obtain an n^{th} association feature of the atom corresponding to the another atom; and perform the following processing in a case that the quantity of the other atoms is multiple: performing summation processing on n^{th} association features of the atom corresponding to multiple other atoms, to obtain an n^{th} association feature of the atom; and performing second fusion processing on the (n−1)^{th} attribute feature and the n^{th} association feature of the atom, to obtain the n^{th} attribute feature of the atom.

In some embodiments, before energy error prediction processing is performed on the three-dimensional structure of the target molecule by invoking the neural network model to obtain the energy error of the target molecule, the apparatus further includes: The training module 4555 is configured to: obtain a sample molecule, and perform conformation generation processing on the sample molecule to obtain multiple sample molecular conformations; obtain a label energy error of each sample molecular conformation; perform forward propagation on each sample molecular conformation in an neural network model to obtain a predicted energy error of each sample molecular conformation; determine a comprehensive loss corresponding to the neural network model based on the label energy error of each sample molecular conformation and the predicted energy error of each sample molecular conformation; and perform backward propagation processing on the comprehensive loss in the neural network model to obtain a parameter change value of the neural network model in a case that the comprehensive loss converges, and update a parameter of the neural network model based on the parameter change value.

In some embodiments, the training module 4555 is further configured to: perform the following processing for each sample molecular conformation: perform first energy computation processing on the sample molecular conformation to obtain first energy of the sample molecular conformation; perform second energy computation processing on the sample molecular conformation to obtain second energy of the sample molecular conformation; and obtain a first difference between the second energy of the sample molecular conformation and the first energy of the sample molecular conformation as the label energy error of the sample molecular conformation.

In some embodiments, the first energy computation processing is energy computation processing based on semi-empirical quantum mechanics, and the second energy computation processing is energy computation processing based on density functional.

In some embodiments, the training module 4555 is further configured to: perform the following processing for each sample molecular conformation: determine a first root mean square error of the sample molecular conformation based on the label energy error of the sample molecular conformation and the predicted energy error of the sample molecular conformation; and obtain other sample molecular conformations of the sample molecule than the sample molecular conformation; and perform the following processing for each of the other sample molecular conformations: determine a second difference between the label energy error of the sample molecular conformation and a label energy error of the another sample molecular conformation, and determine a third difference between the predicted energy error of the sample molecular conformation and a predicted energy error of the another sample molecular conformation; perform root mean square processing on the second difference and the third difference to obtain a second root mean square error of the sample molecular conformation corresponding to the another sample molecular conformation; perform summation processing on second root mean square errors of the sample molecular conformation corresponding to multiple other sample molecular conformations, to obtain a third root mean square error of the sample molecular conformation; and perform third fusion processing on first root mean square errors of multiple sample molecular conformations and third root mean square errors of the multiple sample molecular conformations to obtain a comprehensive loss corresponding to the neural network model.

Some embodiments provide a computer program product, where the computer program product includes a computer program or computer-executable instructions, and the computer-executable instructions are stored in a computer-readable storage medium. A processor of an electronic device reads the computer-executable instructions from the computer-readable storage medium, and the processor executes the computer-executable instructions, so that the electronic device performs the artificial intelligence-based molecule processing method in some embodiments.

Some embodiments provide a computer-readable storage medium, storing computer-executable instructions. When the computer-executable instructions are executed by a processor, the processor performs the artificial intelligence-based molecule processing method provided in some embodiments, for example, the artificial intelligence-based molecule processing method shown in FIG. 4A to FIG. 4C.

In some embodiments, the computer-readable storage medium may be a memory such as an FRAM, a ROM, a PROM, an EPROM, an EEPROM, a flash memory, a magnetic surface memory, an optical disc, or a CD-ROM; or may be any device that includes one or any combination of the foregoing memories.

In some embodiments, the computer-executable instructions may be compiled in a form of a program, software, a software module, a script, or code, in any form of a programming language (including a compilation or interpretation language, or a declarative or procedural language), and may be deployed in any form, including being deployed as an independent program or as a module, component, subroutine, or another unit suitable for use in a computing environment.

In some embodiments, the computer-executable instructions may be but are not necessarily corresponding to a file in a file system, and may be stored in a part of a file that stores another program or data, for example, stored in one or more scripts in a Hypertext Markup Language (HTML) document, stored in a single file dedicated to a program under discussion, or stored in a plurality of synchronous files (for example, a file that stores one or more modules, subprograms, or code parts).

In some embodiments, the computer-executable instruction may be deployed on one electronic device for execution, or executed on a plurality of electronic devices located at one location, or executed on a plurality of electronic devices distributed at a plurality of locations and interconnected by using a communication network.

In some embodiments, first energy computation processing is performed on a three-dimensional structure of a target molecule to obtain first energy of the target molecule. Because a speed of first energy computation processing is higher than that of second energy computation processing, a speed of energy computation processing is increased, energy error prediction processing is performed on the three-dimensional structure of the target molecule by using a neural network model to obtain an energy error of the target molecule, and error correction processing is performed on the computed first energy based on the energy error, to obtain second energy of the target molecule. Because the energy error can represent a computation result difference between second energy computation processing with high precision and first energy computation processing with low precision, the computed first energy can be corrected by using an energy error obtained through deep learning prediction, thereby increasing precision of the second energy.

The foregoing embodiments are used for describing, instead of limiting the technical solutions of the disclosure. A person of ordinary skill in the art shall understand that although the disclosure has been described in detail with reference to the foregoing embodiments, modifications can be made to the technical solutions described in the foregoing embodiments, or equivalent replacements can be made to some technical features in the technical solutions, provided that such modifications or replacements do not cause the essence of corresponding technical solutions to depart from the spirit and scope of the technical solutions of the embodiments of the disclosure and the appended claims.

What is claimed is:

1. An artificial intelligence-based molecule processing method, performed by an electronic device, comprising:
obtaining a three-dimensional structure of a target molecule;
calling a neural network model to perform energy error prediction processing on the three-dimensional structure of the target molecule to obtain an energy error of the target molecule, the neural network model being trained by fitting an energy error of a sample molecule;
performing the first energy computation processing on the three-dimensional structure of the target molecule to obtain first energy of the target molecule; and
performing error correction processing on the first energy of the target molecule based on the energy error of the target molecule to obtain second energy of the target molecule,
wherein the energy error of the sample molecule is a difference between computation results obtained based on energy of the sample molecule is separately computed according to two energy computation mechanisms, the two energy computation mechanisms comprising first energy computation processing and second energy computation processing, and
wherein precision of the first energy computation processing is less than precision of the second energy computation processing and a speed of the first energy computation processing is greater than a speed of the second energy computation processing.

2. The artificial intelligence-based molecule processing method according to claim 1, wherein calling the neural network model comprises:
performing feature extraction processing on the three-dimensional structure to obtain an energy error feature of the target molecule; and
performing full connection processing on the energy error feature to obtain the energy error of the target molecule.

3. The artificial intelligence-based molecule processing method according to claim 2, wherein the neural network model comprises N cascaded feature networks, and
wherein performing the feature extraction processing on the three-dimensional structure comprises:
performing initial feature extraction processing on each atom in the three-dimensional structure to obtain an initial feature of each atom; and
performing, based on a value of n being 1≤n≤N−1, n^{th} feature extraction processing on an input to an n^{th} feature network in the N cascaded feature networks by using the n^{th} feature network to obtain an n^{th} feature of each atom, and transmitting the n^{th} feature to an (n+1)^{th} feature network; or
performing, based on the value of n being N, attribute feature extraction processing on an (n−1)^{th} feature of each atom by using the n^{th} feature network to obtain an n^{th} attribute feature of each atom, performing coordinate feature extraction processing on the (n−1)^{th} feature of each atom by using the n^{th} feature network to obtain an n^{th} coordinate feature of each atom, and forming the energy error feature by using the n^{th} attribute feature and the n^{th} coordinate feature of each atom;
wherein a value range of N meets 2≤N, n is an integer whose value increases from 1, and a value range of n meets 1≤n≤N−1;
wherein based on the value of n being 1, the input to the n^{th} feature network is the initial feature of each atom; and
wherein based on the value of n being 2≤n≤N, the input to the n^{th} feature network is the (n−1)^{th} feature of each atom outputted by an (n−1)^{th} feature network.

4. The artificial intelligence-based molecule processing method according to claim 3, wherein performing the initial feature extraction processing comprises:
obtaining an initial attribute feature of each atom in the three-dimensional structure and an initial coordinate feature of each atom in the three-dimensional structure, the initial attribute feature representing attribute information of the atom and the initial coordinate feature representing location information of the atom; and
performing the following processing for each atom of the three-dimensional structure:
obtaining at least one other atom other than the atom in the three-dimensional structure, and obtaining an initial relationship feature between the atom and each of the at least one other atom, the initial relationship feature representing a connection relationship between the atom and the at least one other atom; and
forming the initial feature of each atom based on the initial attribute feature of the atom, the initial coordinate feature of the atom, and the initial relationship feature of the atom.

5. The artificial intelligence-based molecule processing method according to claim 3, wherein performing the n^{th} feature extraction processing on the input to the n^{th} feature network in the N cascaded feature networks comprises:
performing the following processing on each atom by using the n^{th} feature network:
obtaining a plurality of other atoms other than the atom in the three-dimensional structure;
performing first mapping processing on the (n−1)^{th} feature of the atom and an (n−1)^{th} feature of each of the plurality of other atoms to obtain an n^{th} association feature of the atom corresponding to each of the plurality of other atoms; and
performing second mapping processing on the (n−1)^{th} feature of the atom and the n^{th} association feature of the atom corresponding to each of the plurality of other atoms to obtain the n^{th} feature of the atom.

6. The artificial intelligence-based molecule processing method according to claim 5, wherein performing the first mapping processing on the (n−1)^{th} feature of the atom and an (n−1)^{th} feature of each of the plurality of other atoms comprises:
extracting an (n−1)^{th} coordinate feature of the atom, an (n−1)^{th} attribute feature of the atom, and an (n−1)^{th} relationship feature of the atom from the (n−1)^{th} feature of the atom;
extracting an (n−1)^{th} coordinate feature of each of the plurality of other atoms and an (n−1)^{th} attribute feature of each of the plurality of other atoms from the (n−1)^{th} feature of each of the plurality of other atoms; and
performing the following processing for each of the plurality of other atoms:
extracting an (n−1)^{th} relationship feature of the atom for the another atom from the (n−1)^{th} relationship feature of the atom;
obtaining a first feature distance between the (n−1)^{th} coordinate feature of the atom and the (n−1)^{th} coordinate feature of the another atom; and
performing first fusion processing on a square of the first feature distance, the (n−1)^{th} attribute feature of the atom, the (n−1)^{th} attribute feature of the another atom, and the (n−1)^{th} relationship feature of the atom for the another atom, to obtain the n^{th} association feature of the atom corresponding to the another atom.

7. The artificial intelligence-based molecule processing method according to claim 6, wherein performing the second mapping processing on the (n−1)^{th} feature of the atom and the n^{th} association feature of the atom corresponding to each of the plurality of other atoms comprises:
performing summation processing on n^{th} association features of the atom corresponding to multiple other atoms to obtain an n^{th} association feature of the atom;
performing second fusion processing on the (n−1)^{th} attribute feature of the atom and the n^{th} association feature of the atom to obtain the n^{th} attribute feature of the atom;
obtaining a first feature difference between the (n−1)^{th} coordinate feature of the atom and the (n−1)^{th} coordinate feature of each of the plurality of other atoms;
performing linear mapping processing on the n^{th} association feature of the atom corresponding to each of the plurality of other atoms to obtain a weight of each of the other atoms;
performing weighted average processing on first feature differences of multiple other atoms based on the weight of each of the plurality of other atoms to obtain a weighted average result corresponding to the atom;
performing summation processing on the weighted average result of the atom and the (n−1)^{th} coordinate feature of the atom to obtain the n^{th} coordinate feature of the atom; and
using an initial relationship feature of the atom as an n^{th} relationship feature of the atom, and forming the n^{th} feature of the atom by using the n^{th} relationship feature of the atom, the n^{th} attribute feature of the atom, and the n^{th} coordinate feature of the atom.

8. The artificial intelligence-based molecule processing method according to claim 3, wherein performing the attribute feature extraction processing comprises:
extracting, from the (n−1)^{th} feature of each atom, an (n−1)^{th} coordinate feature of the atom, an (n−1)^{th} attribute feature of the atom, and an (n−1)^{th} relationship feature of the atom; and
obtaining, for each atom, other atoms other than the atom in the three-dimensional structure, and performing the following processing for each of the other atoms:
extracting an (n−1)^{th} relationship feature of the atom for the other atom from the (n−1)^{th} relationship feature of the atom, and obtaining a second feature distance between the (n−1)^{th} coordinate feature of the atom and an (n−1)^{th} coordinate feature of the other atom;
performing first fusion processing on a square of the second feature distance, the (n−1)^{th} attribute feature of the atom, the (n−1)^{th} attribute feature of the other atom, and the (n−1)^{th} relationship feature of the atom for the other atom, to obtain an n^{th} association feature of the atom corresponding to the other atom;
performing summation processing on n^{th} association features of the atom corresponding to multiple other atoms, to obtain an n^{th} association feature of the atom; and
performing second fusion processing on the (n−1)^{th} attribute feature of the atom and the n^{th} association feature of the atom, to obtain the n^{th} attribute feature of the atom.

9. The artificial intelligence-based molecule processing method according to claim 1, wherein before calling the neural network model to perform energy error prediction processing on the three-dimensional structure of the target molecule the method further comprises:
obtaining a sample molecule, and performing conformation generation processing on the sample molecule to obtain multiple sample molecular conformations;
obtaining a label energy error of each sample molecular conformation of the multiple sample molecular conformations;
performing forward propagation on each sample molecular conformation in an initialized neural network model to obtain a predicted energy error of each sample molecular conformation;
determining a comprehensive loss based on the label energy error of each sample molecular conformation and the predicted energy error of each sample molecular conformation; and
performing backward propagation processing on the comprehensive loss in the initialized neural network model to obtain a parameter change value of the initialized neural network model in a case that the comprehensive loss converges, and updating a parameter of the initialized neural network model based on the parameter change value.

10. The artificial intelligence-based molecule processing method according to claim 9, wherein obtaining the label energy error of each sample molecular conformation comprises:
performing the following processing for each sample molecular conformation:
performing first energy computation processing on the sample molecular conformation to obtain first energy of the sample molecular conformation;
performing second energy computation processing on the sample molecular conformation to obtain second energy of the sample molecular conformation; and
obtaining a first difference between the second energy of the sample molecular conformation and the first energy of the sample molecular conformation as the label energy error of the sample molecular conformation.

11. The artificial intelligence-based molecule processing method according to claim 9, wherein determining the comprehensive loss comprises:
performing the following processing for each sample molecular conformation:
determining a first root mean square error of the sample molecular conformation based on the label energy error of the sample molecular conformation and the predicted energy error of the sample molecular conformation; and
obtaining other sample molecular conformations of the sample molecule than the sample molecular conformation; and
performing the following processing for each of the other sample molecular conformations:
determining a second difference between the label energy error of the sample molecular conformation and a label energy error of the other sample molecular conformation, and determining a third difference between the predicted energy error of the sample molecular conformation and a predicted energy error of the other sample molecular conformation;
performing root mean square processing on the second difference and the third difference to obtain a second root mean square error of the sample molecular conformation corresponding to the other sample molecular conformation;
performing summation processing on second root mean square errors of the sample molecular conformation corresponding to multiple other sample molecular conformations to obtain a third root mean square error of the sample molecular conformation; and
performing third fusion processing on first root mean square errors of multiple sample molecular conformations and third root mean square errors of the multiple other sample molecular conformations to obtain a comprehensive loss corresponding to the neural network model.

12. An artificial intelligence-based molecule processing apparatus, comprising:
at least one memory configured to store program code; and
at least one processor configured to read the program code and operate as instructed by the program code, the program code comprising:
obtaining code configured to cause at least one of the at least one processor to obtain a three-dimensional structure of a target molecule;
neural network code configured to cause at least one of the at least one processor to call a neural network model to perform energy error prediction processing on the three-dimensional structure of the target molecule, to obtain an energy error of the target molecule, the neural network model being trained by fitting an energy error of a sample molecule;
computation code configured to cause at least one of the at least one processor to perform the first energy computation processing on the three-dimensional structure of the target molecule to obtain first energy of the target molecule; and
correction code configured to cause at least one of the at least one processor to perform error correction processing on the first energy of the target molecule based on the energy error of the target molecule, to obtain second energy of the target molecule,
wherein the energy error of the sample molecule is a difference between computation results obtained based on energy of the sample molecule is separately computed according to two energy computation mechanisms, the two energy computation mechanisms comprising first energy computation processing and second energy computation processing, and
wherein precision of the first energy computation processing is less than precision of the second energy computation processing and a speed of the first energy computation processing is greater than a speed of the second energy computation processing.

13. The artificial intelligence-based molecule processing apparatus according to claim 12, wherein the neural network code is further configured to cause at least one of the at least one processor to:
perform feature extraction processing on the three-dimensional structure to obtain an energy error feature of the target molecule; and
perform full connection processing on the energy error feature to obtain the energy error of the target molecule.

14. The artificial intelligence-based molecule processing apparatus according to claim 13, wherein the neural network model comprises N cascaded feature networks, and
wherein the neural network code is further configured to cause at least one of the at least one processor to:
perform initial feature extraction processing on each atom in the three-dimensional structure to obtain an initial feature of each atom; and
perform, based on a value of n being 1≤n≤N−1, n^{th} feature extraction processing on an input to an n^{th} feature network in the N cascaded feature networks by using the n^{th} feature network to obtain an n^{th} feature of each atom, and transmitting the n^{th} feature to an (n+1)^{th} feature network; or
perform, based on the value of n being N, attribute feature extraction processing on an (n−1)^{th} feature of each atom by using the n^{th} feature network to obtain an n^{th} attribute feature of each atom, performing coordinate feature extraction processing on the (n−1)^{th} feature of each atom by using the n^{th} feature network to obtain an n^{th} coordinate feature of each atom, and forming the energy error feature by using the n^{th} attribute feature and the n^{th} coordinate feature of each atom;
wherein a value range of N meets 2≤N, n is an integer whose value increases from 1, and a value range of n meets 1≤n≤N−1;
wherein based on the value of n being 1, the input to the n^{th} feature network is the initial feature of each atom; and
wherein based on the value of n being 2≤n≤N, the input to the n^{th} feature network is the (n−1)^{th} feature of each atom outputted by an (n−1)^{th} feature network.

15. The artificial intelligence-based molecule processing apparatus according to claim 14, wherein the neural network code is further configured to cause at least one of the at least one processor to:
obtain an initial attribute feature of each atom in the three-dimensional structure and an initial coordinate feature of each atom in the three-dimensional structure, the initial attribute feature representing attribute information of the atom and the initial coordinate feature representing location information of the atom; and
perform the following processing for each atom of the three-dimensional structure:
obtain at least one other atom other than the atom in the three-dimensional structure, and obtain an initial relationship feature between the atom and each of the at least one other atom, the initial relationship feature representing a connection relationship between the atom and the at least one other atom; and
form the initial feature of each atom based on the initial attribute feature of the atom, the initial coordinate feature of the atom, and the initial relationship feature of the atom.

16. The artificial intelligence-based molecule processing apparatus according to claim 14, wherein the neural network code is further configured to cause at least one of the at least one processor to:
perform the following processing on each atom by using the n^{th} feature network:
obtain a plurality of other atoms other than the atom in the three-dimensional structure;
perform first mapping processing on the (n−1)^{th} feature of the atom and an (n−1)^{th} feature of each of the plurality of other atoms to obtain an n^{th} association feature of the atom corresponding to each of the plurality of other atoms; and
perform second mapping processing on the (n−1)^{th} feature of the atom and the n^{th} association feature of the atom corresponding to each of the plurality of other atoms to obtain the n^{th} feature of the atom.

17. The artificial intelligence-based molecule processing apparatus according to claim 16, wherein the neural network code is further configured to cause at least one of the at least one processor to:
extract an (n−1)^{th} coordinate feature of the atom, an (n−1)^{th} attribute feature of the atom, and an (n−1)^{th} relationship feature of the atom from the (n−1)^{th} feature of the atom;
extract an (n−1)^{th} coordinate feature of each of the plurality of other atoms and an (n−1)^{th} attribute feature of each of the plurality of other atoms from the (n−1)^{th} feature of each of the plurality of other atoms; and
perform the following processing for each of the plurality of other atoms:
extract an (n−1)^{th} relationship feature of the atom for the another atom from the (n−1)^{th} relationship feature of the atom;
obtain a first feature distance between the (n−1)^{th} coordinate feature of the atom and the (n−1)^{th} coordinate feature of the another atom; and
perform first fusion processing on a square of the first feature distance, the (n−1)^{th} attribute feature of the atom, the (n−1)^{th} attribute feature of the another atom, and the (n−1)^{th} relationship feature of the atom for the another atom, to obtain the n^{th} association feature of the atom corresponding to the another atom.

18. The artificial intelligence-based molecule processing apparatus according to claim 17, wherein the neural network code is further configured to cause at least one of the at least one processor to:
perform summation processing on n^{th} association features of the atom corresponding to multiple other atoms to obtain an n^{th} association feature of the atom;
perform second fusion processing on the (n−1)^{th} attribute feature of the atom and the n^{th} association feature of the atom to obtain the n^{th} attribute feature of the atom;
obtain a first feature difference between the (n−1)^{th} coordinate feature of the atom and the (n−1)^{th} coordinate feature of each of the plurality of other atoms;
perform linear mapping processing on the n^{th} association feature of the atom corresponding to each of the plurality of other atoms to obtain a weight of each of the other atoms;
perform weighted average processing on first feature differences of multiple other atoms based on the weight of each of the plurality of other atoms to obtain a weighted average result corresponding to the atom;
perform summation processing on the weighted average result of the atom and the (n−1)^{th} coordinate feature of the atom to obtain the n^{th} coordinate feature of the atom; and
use an initial relationship feature of the atom as an n^{th} relationship feature of the atom, and form the n^{th} feature of the atom by using the n^{th} relationship feature of the atom, the n^{th} attribute feature of the atom, and the n^{th} coordinate feature of the atom.

19. The artificial intelligence-based molecule processing apparatus according to claim 14, wherein the neural network code is further configured to cause at least one of the at least one processor to:
extract, from the (n−1)^{th} feature of each atom, an (n−1)^{th} coordinate feature of the atom, an (n−1)^{th} attribute feature of the atom, and an (n−1)^{th} relationship feature of the atom; and
obtain, for each atom, other atoms other than the atom in the three-dimensional structure, and perform the following processing for each of the other atoms:
extract an (n−1)^{th} relationship feature of the atom for the other atom from the (n−1)^{th} relationship feature of the atom, and obtain a second feature distance between the (n−1)^{th} coordinate feature of the atom and an (n−1)^{th} coordinate feature of the other atom;
perform first fusion processing on a square of the second feature distance, the (n−1)^{th} attribute feature of the atom, the (n−1)^{th} attribute feature of the other atom, and the (n−1)^{th} relationship feature of the atom for the other atom, to obtain an n^{th} association feature of the atom corresponding to the other atom;
perform summation processing on n^{th} association features of the atom corresponding to multiple other atoms, to obtain an n^{th} association feature of the atom; and
perform second fusion processing on the (n−1)^{th} attribute feature of the atom and the n^{th} association feature of the atom, to obtain the n^{th} attribute feature of the atom.

20. A non-transitory computer-readable storage medium storing computer code which, when executed by at least one processor, causes the at least one processor to at least:
obtain a three-dimensional structure of a target molecule;
call a neural network model to perform energy error prediction processing on the three-dimensional structure of the target molecule to obtain an energy error of the target molecule, the neural network model being trained by fitting an energy error of a sample molecule;
perform the first energy computation processing on the three-dimensional structure of the target molecule to obtain first energy of the target molecule; and
perform error correction processing on the first energy of the target molecule based on the energy error of the target molecule to obtain second energy of the target molecule,
wherein the energy error of the sample molecule is a difference between computation results obtained based on energy of the sample molecule is separately computed according to two energy computation mechanisms, the two energy computation mechanisms comprising first energy computation processing and second energy computation processing, and
wherein precision of the first energy computation processing is less than precision of the second energy computation processing and a speed of the first energy computation processing is greater than a speed of the second energy computation processing.
